(19) United States
(12) Patent Application Publication
(10) Pub. No.: US 2025/0302353 A1
(43) Pub. Date: Oct. 2, 2025

(54) SYSTEM FOR TESTING AND TRAINING A BRAIN CAPABILITY AND METHOD OF IMPLEMENTING THE SAME

(71) Applicant: i-BrainTech Ltd, Tel Aviv-Yafo (IL)

(72) Inventors: Konstantin SONKIN, Tel-Aviv (IL); Yoav Zeev ZAMIR, Tel-Aviv (IL)

(73) Assignee: i-BrainTech Ltd, Tel Aviv-Yafo (IL)

(21) Appl. No.: 18/862,183

(22) PCT Filed: May 3, 2023
(86) PCT No.: PCT/IL2023/050453

§ 371 Date: Nov. 1, 2024

Related U.S. Application Data
(60) Provisional application No. 63/337,724, filed on May 3, 2022.

(51) Int. Cl. A61B 5/16; A61B 5/00; A61B 5/256; A61B 5/372; A61B 5/375; A61B 5/378; A61B 5/384; G16H 20/70

(52) U.S. Cl. CPC A61B 5/165; A61B 5/256; A61B 5/372; A61B 5/375; A61B 5/378; A61B 5/384; A61B 5/7405; A61B 5/744; G16H 20/70; A61B 2503/10; A61B 2503/20

(57) ABSTRACT

A system and method is disclosed for testing and training a brain capability of planning and executing motion activity. The system comprises the following: an electroencephalographic sensor arrangement attachable to a head of said trainee; a processor configured for receiving and analyzing electroencephalographic signals obtained from said trainee in response to said visual stimulus displayed to said trainee; a memory storing instructions for
i. instructing the trainee to imagine executing said motion action;
ii. measuring electroencephalographic signals on the electroencephalographic sensor arrangement;
iii. calculating at least one of a concentration index; a motor control index; an alertness index;
iv. and iteratively providing the trainee with a feedback pattern

In some embodiments a display is provided as a visual stimulus to the trainee to which the trainee responds by imagining executing a motion activity.

### FIELD OF THE INVENTION

The present invention generally relates to systems and methods directed to neuro-enhancement of trainees and, more particularly, to neurofeedback training in order to improve a subject's performance.

### BACKGROUND OF THE INVENTION

The use of visualization to train motion capabilities for planning and executing motion 10 activities.

Brain stimulation has been used to train the brain to enhance physical motion activities of the subject. It has been shown that, by applying electric current (externally) over the specific brain regions, physical performance increases. This has been shown, for example, with athletes. During the usage, brain stimulation is quite similar to the result of brain training that 15 is achieved organically with training. The main disadvantage of brain stimulators is the time the effect they carry last. With the device removal, it is reported that the training's impact remains only for ˜20 minutes.

Commercially available technologies for neuro-interventions are devices for mental or cognitive state neurofeedback. These devices are simple to use, sleek-designed EEG sets that allow the customer to train specific brain activation without the need for a professional hardware or operator's help. These products aim to target global and widespread brain processes such as relaxation and stress reduction or concentration. Using auditory or visual feedback, the user can learn how to switch into a relaxed or focused mental state. These devices focus on these general processes as they are the only ones that these headsets can record.

In the current time, these off-the-shelf EEG sets have a low signal to noise ratio, which limits the types of brain activities they can detect and analyze. We expect this to change in the coming years, as technological advancements are racing ahead.

US 2019/0247662 discloses a method of facilitating a skill learning process or improving performance of a task, comprising: determining a brainwave pattern reflecting neuronal activity of a skilled subject while engaged in a respective skill or task; processing the determined brainwave pattern with at least one automated processor; and subjecting a subject training in the respective skill or task to brain entrainment by a stimulus selected from the group consisting of one or more of a sensory excitation, a peripheral excitation, a transcranial excitation, and a deep brain stimulation, dependent on the processed temporal pattern extracted from brainwaves reflecting neuronal activity of the skilled subject.

### Sports

Professional sports are in constant competition. The foundations of almost any sport are rooted in competition. In the competitive environment, sports clubs and professional athletes seek to increase their performance. In addition to the pure value of ‘being the best,’ success in professional sports is also very often a critical factor in the growth of capitalization for the athletes and clubs (KMPG study https://assets.kpmg/content/dam/kpmg/br/pdf/2019/06/the-european-elite-2019a.pdf). Therefore, these clubs are in high and constant need for new technologies that would give their athletes an advantage over competitors. While the sport-tech market is in continuous Market growth (Market and Research; www.marketsandmarkets.com/Market-Reports/sports-technology-market-104958738.html), one of the most promising branches of it is the usage of new Brain-Computer Interface (BCI) developments (Our crowd: Top 10 Tech Trends for 2020, and Beyond—at Summit; https://summit.ourcrowd.com/top-10-tech-trends-for-2020-and-beyond-at-2020-ourcrowdsummit/).

Traditional training, in its core, aims to train the brain by repetition-repeatedly acting until reaching perfection. “Proficient athletes in any sport have practiced the game until the main skills, the sub-skills, and their execution becomes rote”, G. Landrum, “Empowerment: The Competitive Edge in Sports, Business & Life”, 2005. In addition to the fact that the muscles grow with training, the more critical process during practice is the formation of stable and efficient motor plans—rewiring of the brain—that will later bring the action performance into perfection. However, traditional training seemed to help professional athletes to reach a maximal performance level that today is hard to surpass. Some of the main limitations of extensive physical exercise are that they are time-consuming, expensive, and can lead to injuries, exhaustion, or reduce motivation.

Recent breakthrough technologies and scientific discoveries have opened the door to direct reading of brain activity in an ‘online’ manner. The ability to read the brain and analyze its events in real-time has sparked the hope of developing new ways to train the brain, leading to the same physical training results, but that does not suffer from the disadvantages described.

In addition to the constant search for new training methods, an essential shift in the way sports clubs are shaping their training strategy is using new and robust data science tools. These clubs understand the importance of investing their athlete's performance using large amounts of data of different sources (for example—speed, forces, team dynamics, etc.) to extract weak points that could be trained on the one hand or predict future performance on the other. In the sports industry and many other types of industries, it is widely agreed that data is the new gold, and professionals are in a ‘gold rush’ these days. Information about the brain, up until recently, was unavailable and hard to collect.

An excellent example of how brain-data can contribute to sports can be found in the level of concentration an athlete can hold during the match, as it significantly affects his/her performance: “Focusing on what is important is key to effective performance. During play, especially in long games like football, baseball, or soccer, maintaining focus throughout is difficult. Those who keep their focus longest tend to make the fewest errors, giving them an edge” [4]. Traditional measuring of a player's ability to concentrate on his/her task could be done only indirectly either by observing the way he plays or by his/her performance in a specific task that examines concentration (which is not related to the actual sports actions). Neuroscientific research had found that the levels of a person's attention strongly correlate to specific brain patterns that could be measured from external devices (Sok Joo Tan et al., A Brief Review of the Application of Neuroergonomics in Skilled Cognition During Expert Sports Performance, Front. Hum. Neurosci., 2019; https://www.frontiersin.org/articles/10.3389/fnhum.2019.00278/full).

Enhancing performance with direct intervention over brain activity is done commercially in two main ways. The first is brain stimulation. By applying electric current (externally) over the specific brain regions, athletes' performance increases. During the usage, brain stimulation is quite similar to the result of brain training that is achieved organically with training. The main disadvantage of stimulators is the time the effect they carry last. With the device removal, it is reported that the training's impact remains only for ˜20 minutes. It means that in most competitive sports, the advantage of the intervention fades before it is needed. This process can be also considered as a form of doping, which is problematic for competitive sports.

### Testing and Training of Surgeons

About 15 million operating room procedures are performed annually in the U.S. (Weiss & Elixhauser, 2006). A “hotspot” for medical errors, inpatient surgery is associated with 0.4-0.8% rate of death and 3-17% rate of major complications (Haynes et al., 2009). Studies suggest that about half of surgical complications are avoidable (Gawande, Thomas, Zinner, & Brennan, 1999; Kable, Gibberd, & Spigelman, 2002) and high-functioning teams have significant reductions in the number of adverse events (Mazzocco et al., 2009). New techniques that are being introduced potentially improve patient safety but impose dramatic new demands on surgeons' abilities and workload. More than one million laparoscopic surgeries are performed annually in the U.S. where a surgeon operates with an indirect, narrow visual access and minimal tactile feedback. Such conditions require new skills with different learning curves and new training methods beyond the traditional master-apprentice format (Van Hove, Tuijthof, Verdaasdonk, Stassen, & Dankelman, 2010). In fact as healthcare patterns shift toward prevention and quality, previously unexamined aspects of the operating room come into sharper focus and surgeons and trainees are scrutinized for their performance (Kao & Thomas, 2008; Kohn, Corrigan, & Donaldson, 2000; Pavlidis et al., 2012; Risucci, Geiss, Gellman, Pinard, & Rosser, 2001). Thus, there is a long-felt need of providing systems and methods for decoding and measuring the brain patterns during the training of surgical and dental procedures.

### Aviation Testing and Training

According to the Boeing company, 80% of air accidents are caused by human error.

Nasa reported that, during 2004 in the United States, pilot error was listed as the primary cause 20 of 78.6% of fatal general aviation accidents, and as the primary cause of 75.5% of general aviation accidents overall.

Pilot errors may be classified as:
(i) General errors of misjudgment attributable to a variety of causes such as poor training, fatigue, inattention
(ii) Weather related errors such as not fully comprehending the effect of weather on the aircraft in flight.
(iii) Mechanical related errors such as not reacting to an internal fault in the aircraft.

For scheduled air transport, pilot error typically accounts for just over half of worldwide accidents with a known cause.

The recent hiatus in air travel has caused an increase in pilot errors (https://www.latimes.com/business/story/2021-01-29/airline-pilots-flight-errors-pandemic) and only serves to highlight the need for additional and effective systems of training. The system and method of the present invention is easily adaptable to improve pilot's performance and assessment. “There is growing interest for implementing tools to monitor cognitive performance in naturalistic work and everyday life settings. The emerging field of research, known as neuroergonomics, promotes the use of wearable and portable brain monitoring sensors such as functional near infrared spectroscopy (fNIRS) to investigate cortical activity in a variety of human tasks out of the laboratory” (Front. Hum. Neurosci., 2018).

It is a clearly defined need to provide means, systems and methods of training pilots to avoid pilot error.

To summarize, there is a long-felt need of providing systems and methods for decoding and measuring the brain patterns during training tasks such as sporting activities, training of surgeons and aviation testing and training.

### SUMMARY OF THE INVENTION

It is hence one object of the invention to disclose a system for testing and training a brain capability of planning and executing motion activity. The aforesaid system comprises: (a) an electroencephalographic sensor arrangement attachable to a head of said trainee; (b) a processor configured for receiving and analyzing electroencephalographic signals obtained from said trainee in response to said visual stimulus displayed to said trainee; (c) a memory storing instructions when executed by said processor perform: (i) instructing said trainee to imagine executing said sports, surgical or aviation motion action; (ii) measuring electroencephalographic signals on said electroencephalographic sensor arrangement; (iii) calculating at least one characteristic selected from the following: (1) a concentration index; (2) a motor control index; (3) an alertness index; (iv) providing said trainee with a feedback pattern based on at least one said concentration, motor control, alertness and motion readiness; (v) recurring steps c to e if needed.

It is an object of the present invention to disclose the aforementioned system adapted for planning and executing motion activities associated with sport, surgery or aviation or any other activity.

Another object of the invention is to disclose the system comprising a display configured for providing a visual stimulus to said trainee.

A further object of the invention is to disclose the trainee instructed to imagine executing said motion activity in response to displaying said visual stimulus.

A further object of the invention is to disclose the instructions of displaying said visual stimulus, measuring electroencephalographic signals on said electroencephalographic sensor arrangement and providing said feedback pattern performed in a consecutive manner.

A further object of the invention is to disclose the memory comprising an instruction of calculating said concentration index as a ratio of change of electroencephalographic signals at parietal-zone and frontal-zone electrodes at alfa-, beta- and theta-frequencies obtained from said electroencephalographic signals at parietal-zone and frontal-zone electrodes measured at rest.

A further object of the invention is to disclose the memory comprising an instruction of calculating said motor control index as a ratio of change of electroencephalographic signals at sensorimotor zone electrodes, at Mu-frequency obtained from said trainee in response to said visual stimulus below said electroencephalographic signals at sensorimotor zone electrodes measured at rest.

A further object of the invention is to disclose the memory comprising an instruction of for calculating said alertness index as a ratio of change of electroencephalographic signals at parietal-zone electrode at alfa-frequency obtained from said trainee with open eyes over said electroencephalographic signals at parietal-zone with closed eyes.

A further object of the invention is to disclose the memory comprising an instruction of analyzing at least one of said concentration index, motor control index and alertness index of said trainee or a group of said trainees and presenting training progress data in a chronological manner.

A further object of the invention is to disclose the feedback pattern selected from the group consisting of a static avatar, a dynamic avatar, a text message, a sound pattern, a tactile pattern and any combination thereof.

A further object of the invention is to disclose the feedback pattern relating to a visual environment related to said motion action.

A further object of the invention is to disclose the visual environment selected from the group consisting of a soccer stadium, a baseball stadium, a basketball hall, a rugby stadium, an athletic stadium and any combination thereof.

A further object of the invention is to disclose the memory comprising an instruction of calculating an integral index of sports readiness as a compound of at least two indexes selected from the group consisting of said concentration index, motor control index and alertness index and normalized by a sum thereof.

A further object of the invention is to disclose method of testing and training a brain capability of a trainee to plan and executing motion activity. The aforesaid method comprises steps of: (a) providing said system according to claim 1 for testing and training a brain capability of planning and executing sports, surgical or motion actions; (b) instructing said trainee to imagine executing said motion action; (c) measuring electroencephalographic signals on said electroencephalographic electrode arrangement; (d) calculating said concentration index, motor control index and alertness index; e) providing said trainee with a feedback pattern characterizing at least one of said concentration, motor control, alertness and sports readiness; and (f) recurring steps c to e if needed.

A further object of the invention is to disclose the method comprising a step of providing a display configured for providing a visual stimulus to said trainee.

A further object of the invention is to disclose the steps of displaying said visual stimulus, measuring electroencephalographic signals on said electroencephalographic sensor arrangement and providing said feedback pattern which are performed in a consecutive manner.

A further object of the invention is to disclose the step of calculating said concentration index comprising calculating a ratio of change of electroencephalographic signals at parietal-zone and frontal-zone electrodes at alfa-, beta- and theta-frequencies obtained from said trainee in response to said visual stimulus over said electroencephalographic signals at parietal-zone and frontal-zone electrodes measured at rest.

A further object of the invention is to disclose the step of calculating said motor control index comprising calculating a ratio of change of electroencephalographic signals at sensorimotor zone electrodes at Mu-frequency obtained from said trainee in response to said visual stimulus below said electroencephalographic signals at sensorimotor zone electrodes measured at rest.

A further object of the invention is to disclose the step of calculating said alertness index comprising calculating a ratio of excess of electroencephalographic signals at parietal-zone electrode at alfa-frequency obtained from said trainee with open eyes over said electroencephalographic signals at parietal-zone with closed eyes.

A further object of the invention is to disclose the method comprising a step of analyzing at least one of said concentration index, motor control index and alertness index of said trainee or a group of said trainees and presenting training progress data in a chronological manner.

A further object of the invention is to disclose the method comprising a step of calculating an integral index of sports readiness, surgical readiness or pilot readiness as a compound of at least two indexes selected from the group consisting of said concentration index, motor control index and alertness index and normalized by a sum thereof.

### DETAILED DESCRIPTION OF THE INVENTION

The following description is provided, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide systems and methods for testing and training a brain capability of an trainee to plan and execute physical actions. The trainee referred to herein can be a sportsman or woman, surgeon or surgical intern, or pilot or student pilot or anyone engaged in improving or being tested for physical actions.

### Testing and Training in Sports

The systems and methods of the present invention are able to furnish coaches and athletes with relevant information for designing of optimal training protocols (e.g., how long can a player maintain high focus; what are the periodic changes of it during the day/week/month, etc.). Besides, the information relating to athletes' brain states of different traits (e.g., concentration, motor cortex capacity) can be helpful for coaches in determining conditions of trainee athletes or team players and establishing competition or game strategy or in scouting process of new team members.

Nowadays, sports technologies mainly target athletes' performance through physical fitness, flexibility, and other body training types. Until recently, the primary organ that controls movement, which is the brain, was just unreachable, at least, to train directly. This fact had set a limit to the extent to which regular training can contribute to the athlete's improvement. It is essential to understand that the brain controls each movement element from planning to execution. Before almost any type of movement (to exclude spinal reflexes), the mind prepares a motor plan-which muscles to contract, what order, and what intensity. This motor plan determines how well the actual movement will serve the final goal of the sportsmen.

The user's benefit from using a solution based on the current invention includes: (1) performance enhancement, (2) improved return to play after injuries, and (3) a unique approach to new personal Key Performance Indicators KPIs.

By means of short training session once or several times every week, the user learns at first how to enhance specific brain patterns that control his/her movement. In this case, the brain networks' readiness potential is faster to react when needed-which can lead to an increase in performance rate and accuracy. The main advantages of neurofeedback training emerge with time-constant adaptations of these networks come in reshaping these neural networks. As mentioned above, these neural changes are similar to the natural changes that follow traditional training but are performed in higher intensity without the risk of injuries or fatigue.

Different performance measurements are summed up at the end of each training session and presented to the user-what are his/her strong points and his/her weak ones, the dynamics of his/her performance over time, and his/her preferred field or goal positions.

The player can also observe his/her performance over different sessions, track his/her performance in the game, and compare it to his/her in-the-field performance.

The present invention differs from the prior in providing long-term effects which are data-driven. Brain performance is enhanced by learning/training rather by using external stimuli. It should be emphasized that any type of learning/training provides the effects, which last a prolonged period while the effects of the external stimuli are limited by a very short time. The feedback provided by the system of the preset invention relates to the current brain state of the tested trainee. From the abovementioned feedback, the trainee can learn how to control the level of activation of these specific brain regions with their own will. These changes are saved in the brain the same way as in any learning process.

The system of the present invention is configured for recording electroencephalographic signals relating to activation of particular brain regions related to movement such as the primary motor cortex. This is the reason for the use of high-quality EEG hardware—to ensure optimal data collected. By collecting this unique type of data from the user, analyzing it online and offline, we create a training environment that is highly personal which drives the user to increase their performance.

Generally, the system of the present invention is functioning as a Brain-Computer Interface (BCI) for sports and training and includes the following elements: (1) a Brain signal recording arrangement, (2) real-time signal analysis software, and (3) a user's front end (training environment in game form). Data is available on the “brain at work” (Panas aman and Rizzo.

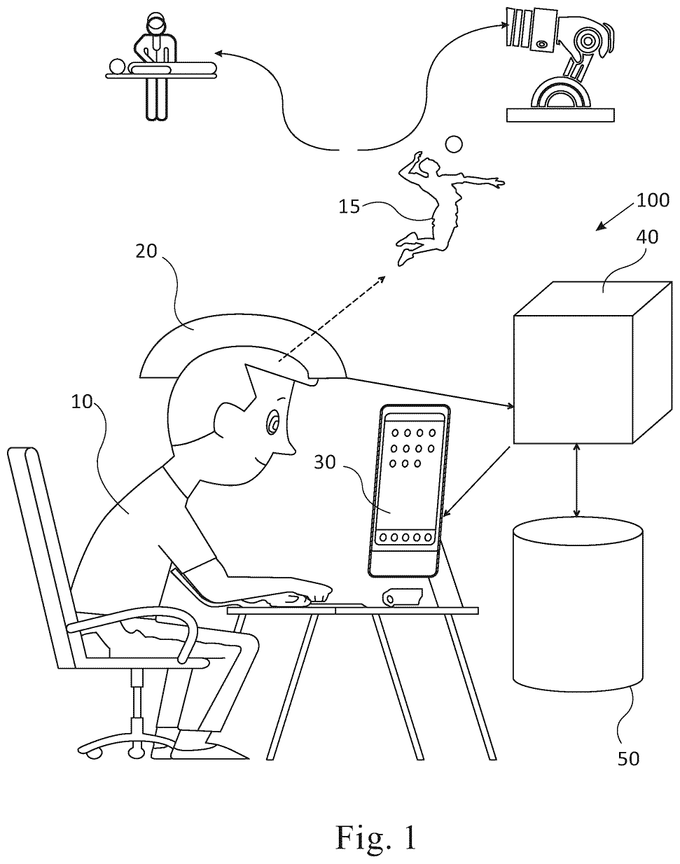

Reference is now made to FIG. 1 presenting a schematic diagram of system for testing and training a brain capability of planning and executing sports actions. Numeral 10 refers to an trainee to be tested. System 100 comprises memory unit 50 storing instructions for processing unit 40. The aforesaid processing unit is connected to electroencephalographic sensor arrangement 20 attachable to a head of said trainee such electroencephalographic signals generated on the head surface of trainee 10 are detectable. Trainee 10 is instructed to imagine executing a predetermined sports action 15 in response to displaying a visual stimulus on display 30. Displaying the visual stimulus to trainee 10 is performed concurrently with measuring electroencephalographic signals on electroencephalographic sensor arrangement 20. According to the instructions stored in memory unit 50, processing unit calculates a concentration index, a motor control index and an alertness index (described in in detail below). Then a feedback message characterizing at least one said concentration, motor control, alertness and sports readiness are provided to trainee 10. The feedback in the form of a static avatar, a dynamic avatar, a text message, a sound pattern, a tactile pattern is in the scope of the present invention.

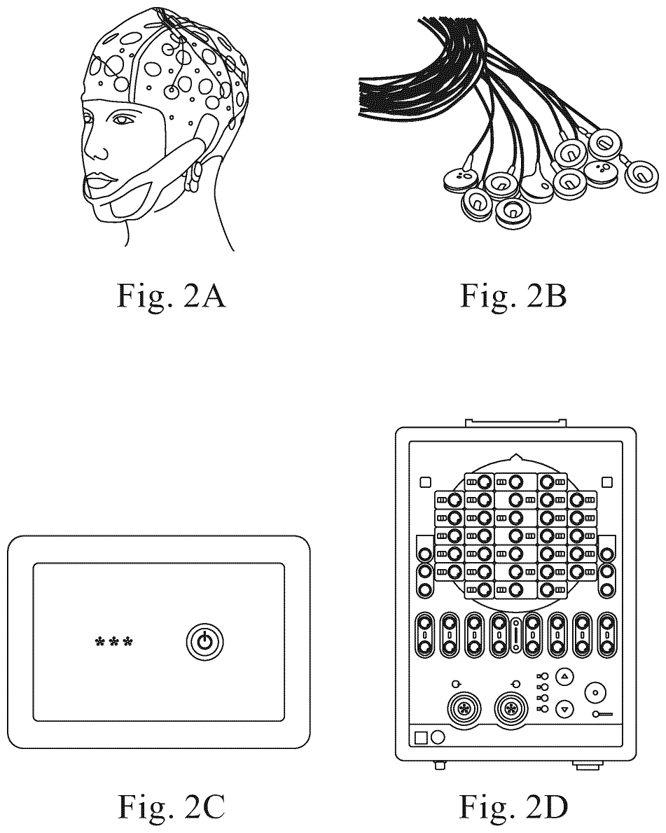

Referring to FIG. 2a, an exemplary electroencephalographic cap is made of an elastic synthetic fabric that comes in various sizes. The aforesaid cap holds the sensors exactly above the brain regions of interest. The standard EEG cap is known as the “10-20 system”. The “International 10-20 system” is a recognized method to describe the scalp electrodes' location. This standard testing system ensures a subject's study outcomes (clinical or research) could be compiled, reproduced, and effectively analyzed and compared using the scientific method. The system is based on the relationship between the location of an electrode and the underlying area of the brain, specifically the cerebral cortex.

Referring to FIG. 2b, in the electroencephalographic sensor arrangement, each EEG sensor is recording the electric fields that are underneath it. The neurons communicate with one another with changes in electric charges. It creates a difference in the electric field around them. That is the reason we can decode the brain's functions by analyzing these changes. The electroencephalographic sensor arrangement comprises sensors attachable at:
(1) frontal zone electrode locates on the midline of the frontal lobe
(2) parietal zone electrode locates on the midline of the parietal lobe;
(3-5) sensorimotor zone electrodes attached to the motor cortex.

In addition, ground and reference sensors are used to collect the signal.

The surface under the sensors is recommended to be cleaned prior to measurement and brought in electric contact the skin surface by means of a conductive gel in order to maximize signal-noise ratio, if necessary.

Referring to FIG. 2c, the sensors are connected to an amplifier that increases the electric signal's magnitude. The amplifier is attached to the cap using a cord. It transmits the amplified electroencephalographic signals to a computer, smartphone, or tablet connected by a USB connector using a wireless connection. Most of commercially available EEG signal amplifiers are usable in the present invention.

The EEG's data is streamed to the processing unit and structured with software according to a pre-set sensor montage. Referring to FIG. 2d, at the next step noise removal features are applied, such as removing the electric network's static electric disturbances (notch filter). The software allows the user to examine each sensor's connectivity level, which will enable them to add more gel if the impedance is too high or detect any sensor malfunctioning.

The EEG data is being streamed using LSL protocol (Lab Streaming Layer), picked up by the data-analyzing code.

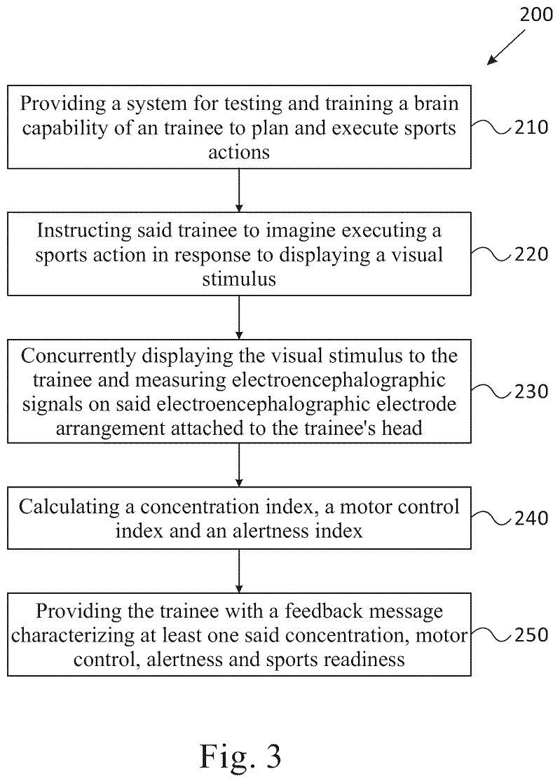

Reference is now made to FIG. 3 presenting method 200 of for testing and training a brain capability of planning and executing sports actions. Method 200 starts with providing system 100 (FIG. 1) for testing and training a brain capability of planning and executing sports actions described above (step 210). After instructing said trainee to imagine executing the predetermined sports action in response to displaying a visual stimulus (step 220), the aforesaid visual stimulus is displayed to the trainee to be tested concurrently with measuring electroencephalographic signals on the electroencephalographic sensor arrangement (step 230). The obtained electroencephalographic signals are processed and the concentration index, motor control index and alertness index are calculated (step 240). On the basis of the calculated indexes, a feedback message characterizing at least one of concentration, motor control, alertness and sports readiness (step 250). Steps 220-250 are recurred if needed.

EEG data is received over an LSL socket. The data analysis includes filtration. The EEG raw-data is analyzed in time windows with a shift (e.g., 500 or 1,000 samples with 50% shift) in each cycle. The goal of the analysis is to extract relevant brain function features that contribute to successful performance of motion control

The level of a person's concentration and activity level in the motor cortex can be deducted from fluctuations of the power of certain frequency bands. We detect it with at least five scalp electrodes.

Calculating of the “concentration” (or—brain engagement) index is performed according to the following algorithm:

Raw data is first filtered using an IIR filter, with half-power frequencies for a frequency range [alpha: 8 to 11 Hz; beta: 16-22 Hz; and theta 4-7 Hz] on the data from sensors attached to parietal zone (alpha) and frontal zone (beta and theta). We calculate power of each frequency band (alpha, beta, and theta) using a “bandpower” method based on the relevant filtered data. The concentration index for each cycle is the ratio of the powers of beta, theta and alpha.

Threshold-accuracy: At the beginning of each session, the system will determine a baseline that characterizes each trainee. The trainee will sit still in front of an instructed simulation for several minutes (2 minutes by default) to create an open-eyes baseline. The system collects the indices during the baseline collection and is used to set an trainee threshold. That would set the user's customized boundary. If exceeded, the system can determine that the user's concentration is high enough to give him positive feedback.

According to an exemplary embodiment of the present invention, the aforesaid threshold can be set as a sum of values of lower bound and a compound of difficulty level and difference between upper and lower bounds

Upper bound is the average of the indices. Lower bound is the average minus two standard deviations of the index, and the Difficulty level is a value set by the user to adjust the challenge level to the user's abilities.

Training using in-game feedback: During the game, the indices are continuously calculated and compared with the trainee's threshold baseline through the backend module. Suppose the concentration index's current value exceeds the threshold set for it. In that case, the module will send the game simulation a message) to grant positive user feedback. As an example, in the soccer game, the user will notice it through shrinkage of a circle indicating the target of the kick, making the shot more accurate. After several attempts during which the user exceeded the threshold in the current trial (for example, in the soccer game, at least five times within 8 seconds), intermittent feedback is given (in the soccer game—the ball would be kicked accurately towards the target).

The algorithm of calculation of the motor control index is the following:

First, raw data is filtered using an IIR filter, with half-power frequencies for a frequency range [Mu: 12 Hz to 15 Hz] for sensorimotor zone sensors, such as C3, Cz and C4 above the motor cortex. The bandpower function calculates the power of the Mu frequency band and the filtered data from these channels. The index from sensorimotor zone sensors, such as C3 and C4 is later used to evaluate current Mu desynchronization which is an action preceding movement.

Threshold-Kick power: all Mu power indices calculated during the acquisition of open-eyes baseline data from the sensorimotor zone, such as C3 and C4 electrodes are used to evaluate the trainee pattern of Mu rhythm of the specific user.

For example, the average Mu power of locations C3 and C4 (above the left motor cortex that controls the right-side limbs and vice versa) is used as the motor brain activity threshold.

Training on the basis of in-game feedback: during the game the current Mu power is continuously compared with the average value of the data collected in the open-eyes baseline. Specifically, the feedback is defined as positive if the sum of momentary Mu power and compound of difficulty lever and STD of MU at baseline is smaller than the mean MU at baseline.

“Mu power” represents the momentary Mu value; Difficulty level is a value set by the user to adjust the challenge level to the user's abilities; STD of Mu at baseline is the standard deviation of all Mu indices collected at open-eyes baseline. Suppose the current Mu power plus a portion of the standard deviation is lower than the average Mu at baseline. In that case, there is an activation of the motor cortex- and positive feedback is given.

In the soccer game training, the circular bar is gradually becoming full, and the color changes from red to green (via yellow and orange). The fill of the bar indicates to the user how to perform the neural action of activating these regions better. If the user succeeds in “charging” this bar during one trial, then the kick's power would be strong enough to score. The similar criteria are applicable to training and scouting in other sports games requiring quick reaction and shooting accuracy such as basketball, hockey, golf, American football, and other. Ability to mobilize physical vigor and mental alertness is also significant to be estimated in training process in different athletic disciplines such as long/high jumps and javelin/hammer/discus throwing.

Only if the user could control both types of feedback (motor control and concentration) simultaneously to a sufficient degree-then the avatar (soccer player on the screen) will successfully score the goal.

The algorithm of calculation of the alertness (sleep) index is the following:

The power of the alpha band in the central parietal sensor, such as Pz, is collected at closed-eyes baseline (several minutes (2 minutes by default), immediately after open-eyes baseline collection) and compared to the alpha power open eyes baseline. Alpha power at this location is known as relating to the user's level of arousal; higher alpha power is usually associated with tiredness.

Threshold-Sleep detection: A moving average for both open-eyes and closed-eyes baseline data is calculated and compared. First, it is based on averaging of one time-window, then the interval increases until the moving average of closed eyes is at least higher in 2 standard deviations than the alpha values at open-eyes state. The moving average with the minimal time interval size that satisfies this condition is set as the threshold for “sleep detection”.

Feedback: During the game, the current alpha power in the central parietal sensor is continuously compared to the threshold. If it exceeds the threshold, a warning sign is sent (in the soccer game's case—the digits of the countdown are painted red). If sleep index was detected a few times during one trial, the trial is disqualified (in the soccer game—the player either misses the ball with his leg or even faint on the grass, a voice message stating “Hey! Wake up!”).

As mentioned, the soccer game is only the first example—but the same concept would be applied in different gaming and training environments.

Using data from early adopters in the field, advanced ML tools can be used, such as clustering algorithms, SVM classifiers, and Artificial Neural Networks to create a powerful pattern detection mechanism that would be highly user-specific, robust to EEG noise and would provide users with rich data-driven insights.

The final element of the system the present invention is the training environment for the user. The interface exposes the user to a real-time representation of his brain activity. It is essential in a neurofeedback learning process and is closing the loop that began in the brain data acquired using the EEG. We designed the products to allow athletes to train brain functions that are crucial to improve their performances. Thus far, we presented the underlying brain processes that our system tracks-motor imagery under high concentration levels. These brain processes are shared between different kinds of sports. However, current research indicates that a feedback environment for this kind of neural training is much more efficient in a proper learning environment. Therefore, for each type of sport, unique virtual environments should be developed. The goal is to allow people to train in a familiar environment, which will enable them to transfer their learning efficiently into the ‘real-world’, such as sports competitions for athletes.

The interface segment of the present invention is a computer-game environment designed to be highly contextual. For example, our first environment is a football (soccer) trainer, and the first task is to kick free shots to the goal.

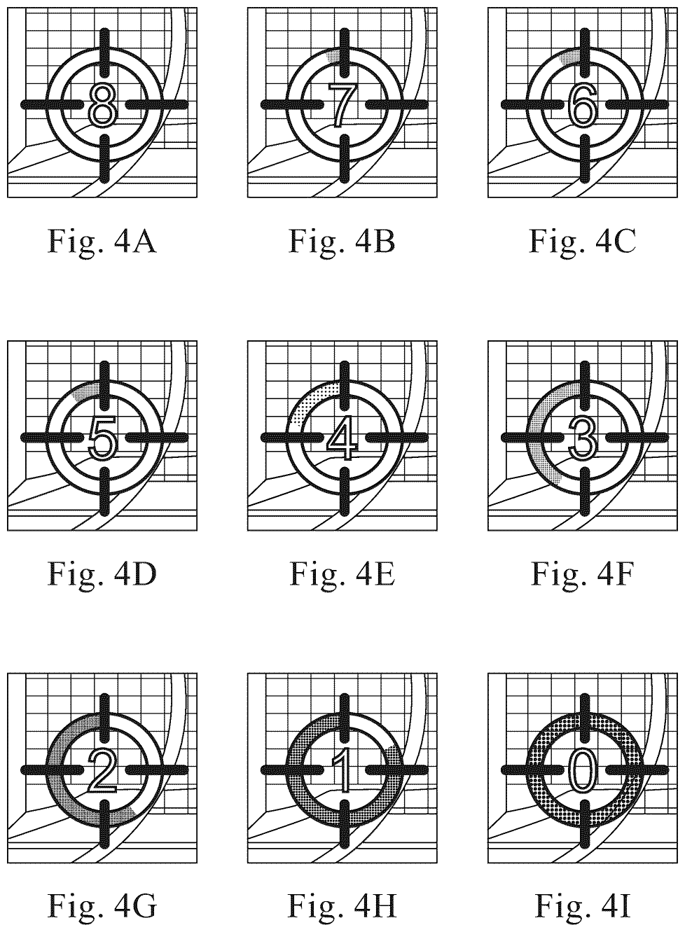

Reference is now made to FIGS. 4a to 4i presenting an exemplarily changing visual stimulus with improving the tested index. Specifically, the trainee is instructed to sit still, focus on the monitor, and imagine that he or she is kicking the ball to a target set on the screen. For each attempt, the system gives the player 8 seconds. The neural activity in the case of real and imagery ‘kick’ is very similar. If our system detects strong activation on specific bands in specific brain areas related to this action, an indicator of kick power will increase (image below). This correlation between neural activation and a symbol presented on the screen allows the user to gain intuitive control of the activity of this neural network that is most important in controlling his leg during a kick.

With practice, the user learns how to control these brain areas, but he is also inducing changes in the way they are ‘wired’. The brain is an organ that changes continuously, neuron connectivity changes, networks of neurons are created or enhanced when constant activation takes place. These changes are the building blocks of learning, and they are the reason we get better with training and repetition.

As the player imagines the action of kicking the ball, the trainee receives continuous feedback on this activity. High activation is indicated by the fill and color of the circular bar (8 is still empty, and 0 is full power), the digit in the middle of the circle is the countdown, telling the user when the kick will take place. Only when the user can reach sufficient power—the avatar will have a chance to score the goal.

The task of activating these networks with repetitive training using our brain-trainer leads to similar actual, in-the-field activity, but does not suffer from many disadvantages of traditional practice.

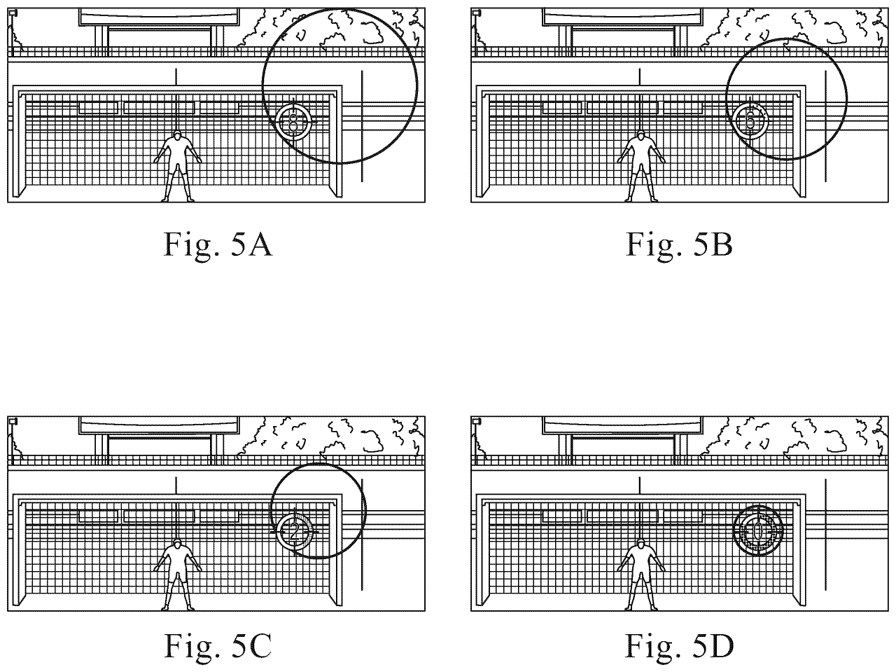

Reference is now made to FIGS. 5a to 5d presenting another embodiment of the present invention. The trainee can control in this game is how accurate the kick will be. The kick accuracy is controlled by the level of engagement and focus of the player. When the system recognizes that the user is focused—a circle around the target will gradually lock on the target. Only when the white circle is locked on the target—the ball will be shot to the goal frame.

This way, the user gets ongoing feedback regarding two mental processes that he could not sense otherwise. The second form of feedback is referred to as ‘intermittent feedback’—the result of their efforts—if they managed to control both types of input to a sufficient level—the kick that will be taken at the end of the 8 seconds will be successful, and they would score. If only one condition is satisfied, then the player misses. If the player could not reach sufficient concentration level during this trial (1 kick), then the ball would be kicked not accurately. If the user could not get enough power by the motor imagery attempts—the kick would be too weak, and the goalkeeper will stop it.

With time and training, the vast majority of users learn how to modulate these neural processes by their own will, leading to important changes in neural networks involved in the player's performance.

If the player reaches sufficient power and accuracy—the avatar will score. Scoring a goal is, by nature, very rewarding for a soccer player. In the brain, a general ‘rule’ is that neural activities that lead to reward will result in stronger connectivity and an increase in resources related to this activity. These changes will increase the player's speed and accuracy in performing motor actions, granting him a competitive advantage.

At the end of the session, the users can observe their performance and track changes in their ability to concentrate or activate the brain's motor areas in the progression of the play.

The interface will allow the coach to design the training session-how many repetitions, locations, which leg is used, etc. Later the athlete and the coach can observe the performance the player achieved in the recent session, compare it to past sessions or other important analyses using a specially designed interface that is presented at the end of a session. It shows statistics based on the results of the training. It allows the users, coaches, or supervisors to have the overall picture of the player's abilities and improvement using our trainee brain-data.

The objective of this invention is to create a basis for development of a series of neuro-interface applications for different sports, recovery, and training applications that will allow users to improve their motor performance. These products will be designed with attentiveness to the needs of every target group of users. The procedure of the present invention is applicable to basketball, American football, hockey, racing, golf, tennis, and other sports, as well as to rehabilitation process.

### Testing and Training of Surgeons

Surgeons use sophisticated instruments for extended periods often under time pressure, communicate with nurses and anesthesiologists, and interact with the complex interfaces of monitors. They possess technical skills acquired through long training. They also deploy an array of non-technical skills (Yule et al., 2008). These include situation awareness (gathering and understanding information and anticipating future states) and task management (responding to change). A strategic action may be, for example, deciding whether to convert a laparoscopic to an open-incision procedure. If the primary tasks (e.g. suturing) present unusual difficulty, this may impair the detection of an important alarm (Frédéric Dehais et al., 2014) or undermine proper planning. Even nearly automated mental processes, such as correcting for camera angle (Klein, Riley, Warm, & Matthews, 2005) or mismatches between the endoscope's optical axis and the instruments' plan on the monitor (Patil, Hanna, & Cuschieri, 2004) may be taking resources away from the surgeon's overall functions. Changes in mental workload due to training or new instrument design will have far reaching implications not only for efficiency but also for patient outcomes. Behavioral and physiological measurements can help improve surgeons' workload monitoring. In developing measures of surgeon workload, hybrid or multi-modal approaches are preferable to unimodal ones, since they are able to deliver greater sets of information that illuminates the operator's functioning from multiple perspectives. Distinct measurement methods often have different strengths and shortcomings and may compensate for each other's artifacts. Furthermore, as hardware becomes increasingly miniaturized and sensor design improves, the cost and effort related to including additional modalities decreases (Gramann et al., 2011). Yurko et al. (2010) utilized NASA-TLX to analyze the laparoscopic performance of novice trainees and to explain the extent of the transfer of their simulator-acquired skill to the operating room (OR). They found that the mental and physical demand ratings obtained at the beginning of training predicted part of the subsequent animal operating room. Such systems and methods are able to furnish surgeons and their trainers and educators with relevant information for designing of optimal training protocols (e.g., how long can a surgeon maintain high focus; what are the periodic changes of it during the day/week/month, etc.). Besides, the information relating to surgeons' brain states of different traits (e.g., concentration, motor cortex capacity) can be helpful for surgical instructors and surgical directors in determining conditions of trainee surgeons or testing aptitude of potential surgeons in embarking on this exacting career requiring very specific manual dexterity skills

Such systems and methods described in the present invention, when appropriately adapted, are able to furnish surgeons and dentists with relevant information for designing of optimal training protocols (e.g., how long can a surgeon maintain high focus; what are the periodic changes of high focus periods during the day/week/month, etc.). Information relating to surgeon's brain states of different traits (e.g., concentration, motor cortex capacity) can be helpful for surgical educators, proctors and supervisors in determining conditions, professional suitability and assessments of trainee surgeons or operating room (OR) personnel and surgical teams.

As with sporting activities, before almost any type of movement used by surgeons or dentists, (to exclude spinal reflexes), the mind prepares a motor plan-which muscles to contract, what order, and what intensity. This motor plan determines how well the actual movement will serve the final goal of the doctor

The surgical PI doctor's benefit from using a solution based on the current invention includes: (1) performance enhancement and (2) a unique approach to new personal Key Performance Indicators KPIs.

KPIs for surgeons can include a well-defined performance measure that is used to observe, analyze, optimize, and transform a surgeon's process to increase satisfaction for both patients and healthcare providers alike. These metrics are commonly used by care facilities to compare their performance to other care facilities and identify areas of improvement. For example, surgical operating room error rate measures the number of mistakes made by the surgeon when treating a patient. The error rate can be represented by (Number of Treatment Errors/Total Treatments)*100

Embodiments of the present invention disclose a system for testing and training a brain capability of planning and executing motion activity. The aforesaid system comprises: (a) an electroencephalographic sensor arrangement attachable to a head of said trainee; (b) a processor configured for receiving and analyzing electroencephalographic signals obtained from said trainee in response to said visual stimulus displayed to said trainee; (c) a memory storing instructions when executed by said processor perform: (i) instructing said trainee to imagine executing said sports motion action; (ii) measuring electroencephalographic signals on said electroencephalographic sensor arrangement; (iii) calculating at least one characteristic selected from the following: (1) a concentration index; (2) a motor control index; (3) an alertness index; (iv) providing said trainee with a feedback pattern based on at least one said concentration, motor control, alertness and motion readiness; (v) recurring steps c to e if needed.

The implementation of system and methods herein disclosed for testing and training a brain capability of planning and executing surgical motion activity can be used to demonstrate construct validity. The system of the present invention can be used to test and train psychomotor, visuo-spatial, and perceptual abilities, and can be used to correlate positively with objective tests of such fundamental abilities that have already been shown to predict surgical performance. Functional involvement of psychomotor ability in the adaptation, consolidation, and development of skills in endoscopic surgery has been demonstrated (Gallagher A G, McClure N, McGuigan J, et al.) An ergonomic analysis of the fulcrum effect in acquisition of endoscopic skills. Endoscopy 1998; 30:617-20 Comprehensive metrics for an entire surgical procedure (endoscopic sinus surgery) have been developed culminating in ES3 developed by Lockheed Martin to teach core ESS procedures to otolaryngology residents (Rudman D T, Stredney D, Sessanna D, et al. Functional endoscopic sinus surgery training simulator. Laryngoscope 1998; 108:1643-7. Edmond C V, Heskamp D, Sluis D, et al. ENT endoscopic surgical training simulator. In: Morgan K S, eds. Medicine meets virtual reality. Amsterdam: IOS Press, 1997:518-28. 25 Wiet G J, Yagel R, Stredney D, et al.) A volumetric approach to virtual simulation of functional endoscopic sinus surgery. Stud Health Technol Inform 1997; 39:167-79).

The ES3 is composed of four principal components: N a Silicon Graphics Incorporated computer which serves as the simulation host platform; N a haptic system controller PC which performs the requisite high rate control of a physical instrument handle associated with a set of virtual surgical instruments; N a virtual voice recognition instructor PC which responds to spoken commands controlling the simulator; N an electro-mechanical platform which houses a physical replica of an endoscope, a mechanically linked surgical hand tool handle, and a mannequin of the external head anatomy

Reducing errors with surgical simulation that are functionally related to tasks carried out in laparoscopic surgery and then receives feedback about performance in these areas:
1. The subject simulates grasping tissue, transferring it from one gripper to the other, running the bowel by using hand over hand transfer, removing a tool from the operating field and reinserting it accurately, cauterising three subtargets, and maintaining objects within the target box while cauterising three consecutive subtargets
2. Visuo-spatial ability This was assessed using the card rotation, cube comparison, and map planning tests from a kit of factor referenced cognitive tests These tests assess the subject's appreciation of the spatial representation of objects that are arranged in various ways.
3. Perceptual assessment This was measured through a test called pictorial surface orientation (PicSOr): Each item is a picture on a computer monitor, showing a spinning arrowhead with its point touching the surface of a cube or a sphere. The subject manoeuvres the arrowhead (using cursor keys) until its shaft is perpendicular to the object's surface at the point where they touch. This is a relatively pure test of a subject's ability to recover the pictorial cues that specify orientation of structures in (virtual) pictorial space, and to compare the implied orientations. The most important measure of performance is the correlation between theoretically correct arrowhead orientation and the setting chosen by the subject, and the slope of the fitted regression line.

Outcomes and predictions of the present system and methods of the invention can be evaluated for internal validity and consistency and with the herein described ES3, and linked with other, standardised measures of cognitive and psychomotor skill for trainee student surgeons. Quantitatively defined types of error can be based on the metrics specified to include:—incorrect manoeuvres, with violation of tissue or instrument tolerances; —correctly performed instrument manoeuvres that are out of sequence or inappropriate for that part of an operation; —inefficient force patterns or application, and inefficient manoeuvres or sequences of manoeuvres; —inappropriate variability in technical performance; —inappropriate “dwell time” or “lack of progress,” indicating indecision or confusion. The present system and method, having been correlated appropriately with standard systems such as the ES3 can be used to provide, contextually accurate analysis and feedback to the student surgeon, for error recognition and correction, in addition to objective comparison

The database is the fundamental unit that integrates the project. The metrics component identified quantifiable measures which then become the fields for the database. The system of the present invention acquires measurements during training and can submit data in an automated and standardised format to a database, which may be web based.

Data may be provided on outcomes that represent an overall assessment of technical skill for an trainee surgeon. These statistics, when assessed together with other archived measures of cognitive and interpersonal skill, may also provide a first order metric for the global assessment of competency. The present system and method may be used to provide data in a recursive, and iterative feedback cycle to support the training and assessment of the surgeons and OR teams Datasets for new operations may be acquired and models may be based on them. Support analysis—based on demographics, training, and performance—across many simulated procedures or groups of surgeons to define parameters of competency, skills, and training for credentialling, regulatory, and policy purposes to appropriate surgical boards and societies, and to state and federal agencies may be implemented.

By means of short training session once or several times every week, the trainee or qualified surgeon learns at first how to enhance specific brain patterns that control his/her movement. In this case, the brain networks' readiness potential is faster to react when needed—which can lead to an increase in performance rate and accuracy. The main advantages of neurofeedback training emerge with time—constant adaptations of these networks come in reshaping these neural networks. As mentioned above, these neural changes are similar to the natural changes that follow traditional training but are performed in higher intensity without the risk of injuries or fatigue.

Different performance measurements are summed up at the end of each training session and presented to the user-what are his/her strong points and his/her weak ones, the dynamics of his/her performance over time, and his/her preferred field or goal positions.

The player can also observe his/her performance over different sessions, track his/her performance in the game, and compare it to his/her in-the-field performance.

The present invention differs from the prior in providing long-term effects which are data-driven. Brain performance is enhanced by learning/training rather by using external stimuli. It should be emphasized that any type of learning/training provides the effects, which last a prolonged period while the effects of the external stimuli are limited by a very short time. The feedback provided by the system of the preset invention relates to the current brain state of the tested trainee. From the abovementioned feedback, the trainee can learn how to control the level of activation of these specific brain regions with their own will. These changes are saved in the brain the same way as in any learning process.

The system of the present invention is configured for recording electroencephalographic signals relating to activation of particular brain regions related to movement such as the primary motor cortex. This is the reason for the use of high-quality EEG hardware—to ensure optimal data collected. By collecting this unique type of data from the user, analyzing it online and offline, we create a training environment that is highly personal which drives the user to increase their performance.

Generally, the system of the present invention is functioning as a Brain-Computer Interface (BCI) for surgical training and includes the following elements: (1) a Brain signal recording arrangement, (2) real-time signal analysis software, and (3) a user's front end (training environment in an OR form).

Reference is now made to FIG. 1 presenting a schematic diagram of system for testing and training a brain capability of planning and executing surgical activities and manoeuvres Numeral 10 refers to an trainee to be tested. System 100 comprises memory unit 50 storing instructions for processing unit 40. The aforesaid processing unit is connected to electroencephalographic sensor arrangement 20 attachable to a head of said trainee such electroencephalographic signals generated on the head surface of trainee 10 are detectable. Trainee 10 is instructed to imagine executing a predetermined surgical action 15 in response to displaying a visual stimulus on display 30. Displaying the visual stimulus to trainee 10 is performed concurrently with measuring electroencephalographic signals on electroencephalographic sensor arrangement 20. According to the instructions stored in memory unit 50, processing unit calculates a concentration index, a motor control index and an alertness index (described in in detail below). Then a feedback message characterizing at least one said concentration, motor control, alertness and sports readiness are provided to trainee 10. The feedback in the form of a static avatar, a dynamic avatar, a text message, a sound pattern, a tactile pattern is in the scope of the present invention.

Referring to FIG. 2a, an exemplary electroencephalographic cap is made of an elastic synthetic fabric that comes in various sizes. The aforesaid cap holds the sensors exactly above the brain regions of interest. The standard EEG cap is known as the “10-20 system”. The “International 10-20 system” is a recognized method to describe the scalp electrodes' location. This standard testing system ensures a subject's study outcomes (clinical or research) could be compiled, reproduced, and effectively analyzed and compared using the scientific method. The system is based on the relationship between the location of an electrode and the underlying area of the brain, specifically the cerebral cortex.

Referring to FIG. 2b, in the electroencephalographic sensor arrangement, each EEG sensor is recording the electric fields that are underneath it. The neurons communicate with one another with changes in electric charges. It creates a difference in the electric field around them. That is the reason we can decode the brain's functions by analyzing these changes. The electroencephalographic sensor arrangement comprises sensors attachable at:
(1) frontal zone electrode locates on the midline of the frontal lobe 25
(2) parietal zone electrode locates on the midline of the parietal lobe;
(3-5) sensorimotor zone electrodes attached to the motor cortex.

In addition, ground and reference sensors are used to collect the signal.

The surface under the sensors is recommended to be cleaned prior to measurement and brought in electric contact the skin surface by means of a conductive gel in order to maximize signal-noise ratio, if necessary.

Referring to FIG. 2c, the sensors are connected to an amplifier that increases the electric signal's magnitude. The amplifier is attached to the cap using a cord. It transmits the amplified electroencephalographic signals to a computer, smartphone, or tablet connected by a USB connector using a wireless connection. Most of commercially available EEG signal amplifiers are usable in the present invention.

The EEG's data is streamed to the processing unit and structured with software according to a pre-set sensor montage. Referring to FIG. 2d, at the next step noise removal features are applied, such as removing the electric network's static electric disturbances (notch filter). The software allows the user to examine each sensor's connectivity level, which will enable them to add more gel if the impedance is too high or detect any sensor malfunctioning.

The EEG data is being streamed using LSL protocol (Lab Streaming Layer), picked up by the data-analyzing code.

Reference is now made to FIG. 3 presenting method 200 of for testing and training a brain capability of planning and executing surgical actions. Method 200 starts with providing system 100 (FIG. 1) for testing and training a brain capability of planning and executing surgical actions described above (step 210). After instructing said trainee to imagine executing the predetermined surgical action in response to displaying a visual stimulus (step 220), the aforesaid visual stimulus is displayed to the trainee to be tested concurrently with measuring electroencephalographic signals on the electroencephalographic sensor arrangement (step 230). The obtained electroencephalographic signals are processed and the concentration index, motor control index and alertness index are calculated (step 240). On the basis of the calculated indexes, a feedback message characterizing at least one of concentration, motor control, alertness and surgical readiness (step 250). Steps 220-250 are recurred if needed.

EEG data is received over an LSL socket. The data analysis includes filtration. The EEG raw-data is analyzed in time windows with a shift (e.g., 500 or 1,000 samples with 50% shift) in each cycle. The goal of the analysis is to extract relevant brain function features that contribute to successful performance of motion control

The level of a person's concentration and activity level in the motor cortex can be deducted from fluctuations of the power of certain frequency bands. In the present system it is detected with at least five scalp electrodes.

Calculating of the “concentration” (or—brain engagement) index is performed according to the following algorithm:

Raw data is first filtered using an IIR filter, with half-power frequencies for a frequency range [alpha: 8 to 11 Hz; beta: 16-22 Hz; and theta 4-7 Hz] on the data from sensors attached to parietal zone (alpha) and frontal zone (beta and theta). We calculate power of each frequency band (alpha, beta, and theta) using a “bandpower” method based on the relevant filtered data. The concentration index for each cycle is the ratio of the powers of beta, theta and alpha.

Threshold—accuracy: At the beginning of each session, the system will determine a baseline that characterizes each trainee. The trainee will sit still in front of an instructed surgical activity or OR simulation for several minutes (2 minutes by default) to create an open-eyes baseline. The system collects the indices during the baseline collection and is used to set an traineeized threshold. That would set the user's customized boundary. If exceeded, the system can determine that the user's concentration is high enough to give him positive feedback.

According to an exemplary embodiment of the present invention, the aforesaid threshold can be set as a sum of values of lower bound and a compound of difficulty level and difference between upper and lower bounds

Upper bound is the average of the indices. Lower bound is the average minus two standard deviations of the index, and the Difficulty level is a value set by the user to adjust the challenge level to the user's abilities.

Training using surgical operation or surgical procedure feedback: During the surgical operation or procedure, the indices are continuously calculated and compared with the trainee's threshold baseline through the backend module. Suppose the concentration index's current value exceeds the threshold set for it. In that case, the module will send the game simulation a message) to grant positive user feedback. As an example, in a suturing, dissection or ablation procedure, the user will notice it through shrinkage of a circle indicating the target of the suturing, dissection or ablation procedure, making the surgical activity more accurate. After several attempts during which the user exceeded the threshold in the current trial (for example, in a surgical procedure a predetermined number of stitches within a predetermined number of seconds), intermittent feedback is given (a satisfactory stitching sequence)).

The algorithm of calculation of the motor control index is the following:

First, raw data is filtered using an IIR filter, with half-power frequencies for a frequency range [Mu: 12 Hz to 15 Hz] for sensorimotor zone sensors, such as C3, Cz and C4 above the motor cortex. The bandpower function calculates the power of the Mu frequency band and the filtered data from these channels. The index from sensorimotor zone sensors, such as C3 and C4 is later used to evaluate current Mu desynchronization which is an action preceding movement.

Threshold—power exerted on the surgical manual instrument: all Mu power indices calculated during the acquisition of open-eyes baseline data from the sensorimotor zone, such as C3 and C4 electrodes are used to evaluate the trainee pattern of Mu rhythm of the specific user.

For example, the average Mu power of locations C3 and C4 (above the left motor cortex that controls the right-side limbs and vice versa) is used as the motor brain activity threshold.

Training on the basis of operation or procedure feedback: during the operation or procedure the current Mu power is continuously compared with the average value of the data collected in the open-eyes baseline. Specifically, the feedback is defined as positive if the sum of momentary Mu power and compound of difficulty lever and STD of MU at baseline is smaller than the mean MU at baseline.

“Mu power” represents the momentary Mu value; Difficulty level is a value set by the user to adjust the challenge level to the user's abilities; STD of Mu at baseline is the standard deviation of all Mu indices collected at open-eyes baseline. Suppose the current Mu power plus a portion of the standard deviation is lower than the average Mu at baseline. In that case, there is an activation of the motor cortex- and positive feedback is given.

In surgical training, the circular bar is gradually becoming full, and the color changes from red to green (via yellow and orange). The fill of the bar indicates to the user how to perform the neural action of activating these regions better. If the user succeeds in “charging” this bar during one trial, then the power exerted on the surgical manual instrument would be strong enough to complete the predetermined surgical action. Similar criteria are applicable to training and selection in all branches of surgery requiring quick and decisive accuracy Ability to mobilize physical vigor and mental alertness is also crucial in the training process for different types of surgery, from general surgery, orthopedic, cardiac, thoracic, neurosurgery, ophthalmic surgery, dental surgery, veterinary surgery and other types of surgical training.

Only if the user could control both types of feedback (motor control and concentration) simultaneously to a sufficient degree-then the avatar (the surgeon on the screen) will successfully complete the procedure.

The algorithm of calculation of the alertness (sleep) index is the following:

The power of the alpha band in the central parietal sensor, such as Pz, is collected at closed-eyes baseline (several minutes (2 minutes by default), immediately after open-eyes baseline collection) and compared to the alpha power open eyes baseline. Alpha power at this location is known as relating to the user's level of arousal; higher alpha power is usually associated with tiredness.

Threshold-Sleep detection: A moving average for both open-eyes and closed-eyes baseline data is calculated and compared. First, it is based on averaging of one time-window, then the interval increases until the moving average of closed eyes is at least higher in 2 standard deviations than the alpha values at open-eyes state. The moving average with the minimal time interval size that satisfies this condition is set as the threshold for “sleep detection”.

Feedback: During the surgical procedure, the current alpha power in the central parietal sensor is continuously compared to the threshold. If it exceeds the threshold, a warning sign is sent (in the case of surgery—the digits of the countdown may be painted red). If sleep index was detected a few times during one trial, the trial is disqualified (in the case of an operation, the surgeon makes an error, the procedure on the screen is incomplete, an alert is given and a voice message may call out “Hey! Wake up!”).

Using data from early adopters in the field, advanced ML tools can be used, such as clustering algorithms, SVM classifiers, and Artificial Neural Networks to create a powerful pattern detection mechanism that would be highly user-specific, robust to EEG noise and would provide users with rich data-driven insights.

The final element of the system the present invention is the training environment for the user. The interface exposes the user to a real-time representation of his brain activity. It is essential in a neurofeedback learning process and is closing the loop that began in the brain data acquired using the EEG. We designed the products to allow student surgeons and surgeons to train brain functions that are crucial to improve their performances. Thus far, we presented the underlying brain processes that our system tracks-motor imagery under high concentration levels. These brain processes are shared between different kinds of surgery. However, current research indicates that a feedback environment for this kind of neural training is much more efficient in a proper learning environment. Therefore, for each type of surgery, unique virtual environments are developed. The goal is to allow people to train in a familiar environment, which will enable them to transfer their learning efficiently into the ‘real-world’ of surgery in the OR.

The interface segment of the present invention is a computer-game surgery environment designed to be highly contextual. For example, our first environment is an intestinal operation where a task might be to excise a growth in the intestine and suture the intestine securely, safely and effectively.

Reference is now made to FIGS. 4a to 4i presenting an exemplarily changing visual stimulus with improving the tested index. Specifically, the surgeon is instructed to sit still, focus on the monitor, and imagine that he or she is excising a target tumour on the screen. For each attempt, the system gives the surgeon a predetermined number of seconds to complete the action. The neural activity in the case of real and imagery excisions is very similar. If our system detects strong activation on specific bands in specific brain areas related to this action, an indicator of power exerted upon the manual surgical instrument will increase (image below). This correlation between neural activation and a symbol presented on the screen allows the user to gain intuitive control of the activity of this neural network that is most important in controlling his hand during the procedure.

With practice, the user learns how to control these brain areas, but he is also inducing changes in the way they are ‘wired’. The brain is an organ that changes continuously, neuron connectivity changes, networks of neurons are created or enhanced when constant activation takes place. These changes are the building blocks of learning, and they are the reason we get better with training and repetition.

As the player imagines the action of excising through, for example, the margin between healthy and tumorous tissue, the trainee surgeon receives continuous feedback on this activity. High activation is indicated by the fill and color of the circular bar (8 is still empty, and 0 is full power), the digit in the middle of the circle is the countdown, telling the user when the irreversible excision should begin. Only when the user can reach sufficient power—the avatar will have a chance to slice the tissue.

The task of activating these networks with repetitive training using our brain-trainer leads to similar actual, in-the-field activity, but does not suffer from many disadvantages of traditional practice.

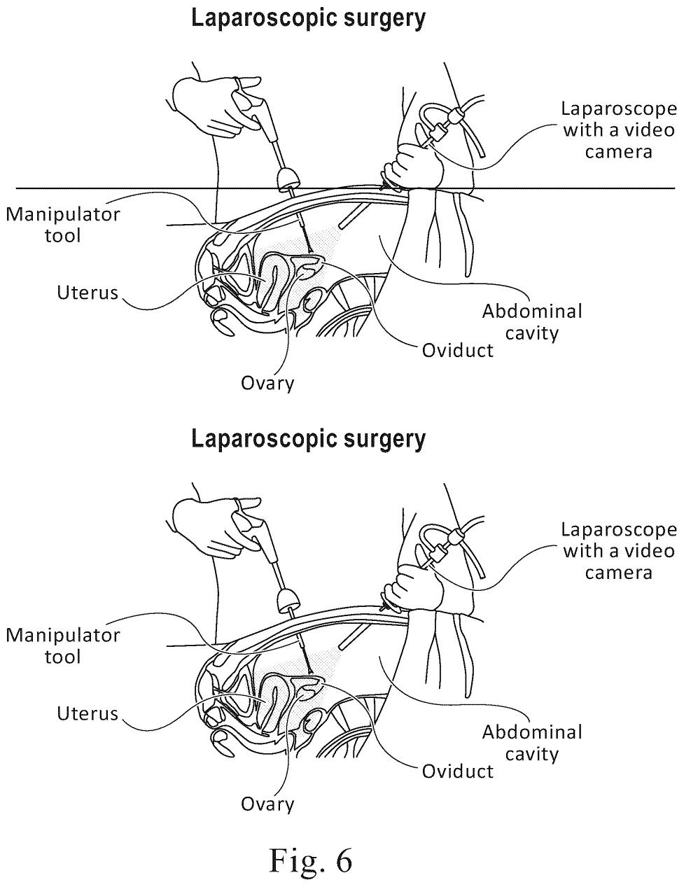

Reference is now made to FIG. 6 presenting another embodiment of the present invention, which could be the operation of surgical devices in laparoscopy surgery. The trainee can control in this game is how accurate the laser beam will be. The accuracy is controlled by the level of engagement and focus of the player. When the system recognizes that the user is focused-a circle around the target will gradually lock on the target. Only when the white circle is locked on the target-will the laser intensity increase to the surgical level required to ablate tissue.

This way, the user gets ongoing feedback regarding two mental processes that he could not sense otherwise. The second form of feedback is referred to as ‘intermittent feedback’—the result of their efforts—if they managed to control both types of input to a sufficient level—the operative manual pressure on the surgical instrument that will be taken at the end of the predetermined time allocation will be successful, and the instrument mediated action would achieve the intended result. If only one condition is satisfied, then the surgeon fails. If the surgeon could not reach sufficient concentration level during this trial then the surgical action would be inaccurately executed. If the user could not exert enough power by the motor imagery attempts—the tissue would not be excised correctly (for example).

With time and training, the vast majority of users learn how to modulate these neural processes by their own will, leading to important changes in neural networks involved in the player's performance.

If the surgeon reaches sufficient power and accuracy—the avatar will complete the surgical procedure and the trainee will be alerted. Successfully completing a crucial surgical operation is very rewarding for the surgeon. In the brain, a general ‘rule’ is that neural activities that lead to reward will result in stronger connectivity and an increase in resources related to this activity. These changes will increase the surgeon speed and accuracy in performing motor actions, providing subjective confidence and objective performance improvement.

At the end of the session, the users can observe their performance and track changes in their ability to concentrate or activate the brain's motor areas in the progression of the operation.

The interface will allow the surgical educators to design the training session-how many repetitions, locations, complications, simulated emergencies are presented. Later the trainee surgeon and the trainer or proctor can observe the performance of the surgeon achieved in the recent session, compare it to past sessions or other important analyses using a specially designed interface that is presented at the end of a session. It shows statistics based on the results of the training. It allows the surgeons under training and the supervisors, educators or proctors to have the overall picture of the surgeon's abilities and improvement using our traineeized brain-data.

### Aviation Testing and Training

It has been proposed to improve the underlying neurocognitive processes of pilots while flying to improve safety and efficiency of the overall human-machine pairing. This could be achieved by (i) the augmentation of human performance and its translation to improved functioning “at work”, (ii) informing the design of the complex systems, or (iii) adapting the user interface and task parameters dynamically during use.

Pilots deal with an uncertain environment and face complex interaction with the flightdeck (Causse et al., 2013; çakir et al., 2016; Reynal et al., 2016). For instance, several studies have emphasized that pilots' working memory (WM) abilities are heavily recruited to handle flightpath, to monitor the flight parameters, and to maintain an up-to-date situation awareness (Causse et al., 2011a,b). WM is also an important component when following air traffic control (ATC) instructions (Morrow et al., 1993). This activity indeed requires mentally storing flight parameters (e.g., heading, altitude, speed) to follow the adequate flight path. However, it is well-known that human working memory is fundamentally limited (Baddeley, 1992; Miller, 1994) and easily overwhelmed when task demand is excessive (Durantin et al., 2014a). Human factor studies emphasized that a variety of environmental stressors may negatively impact pilots' ability to execute ATC clearances (Billings and Cheaney, 1981; Taylor et al., 1994, 2005; Scerbo et al., 2003; Risser et al., 2006; Rome et al., 2012; Dehais et al., 2017). Thus, the implementation of monitoring technology in the cockpit to infer a state of cognitive limitation could represent a promising approach to enhance flight safety (Roy et al., 2017; Verdière et al., 2018).

Indeed, the development of brain computer interface (BCI) technology provides interesting prospects to continuously monitor and take advantage of the brain dynamics and the neural mechanisms underlying cognition. Among the three categories of BCIs (active, reactive, and passive) (Zander and Kothe, 2011; Vecchiato et al., 2016), the first two types are aimed at transforming cerebral activity into messages or commands to voluntarily control distant apparatus (e.g., mouse cursor). Passive BCIs (pBCI) are of particular interest for neuroergonomic applications (Cutrell and Tan, 2008; Frey et al., 2017; Gramann et al., 2017). They allow the use of interpretation of unlabeled brain activity during a task to derive various 30 mental states (Blankertz et al., 2010; Roy et al., 2013; Van Erp et al., 2015; Zander et al., 2017). These mental state-inference systems offer a unique insight into the development of the human-system interactions to overcome cognitive limitations (Zander and Kothe, 2011; Brouwer et al., 2013). While several pBCIs have been successfully implemented in driving (Dijksterhuis et al., 2013) and flight simulator (Gateau et al., 2015; Aricò et al., 2016; çakir et al., 2016; Callan et al., 2016; Verdière et al., 2018), few have attempted to test these systems under more realistic settings. However, very few studies have attempted to test these adaptive systems under realistic settings (Callan et al., 2015).

Electroencephalography (EEG) and functional near infrared spectroscopy (fNIRS) has been gaining popularity recently as the sensors have been miniaturized, become portable and wireless (Ayaz et al., 2013; Strait et al., 2014; Naseer and Hong, 2015; Schudlo and Chau, 2015).

The present invention differs from the prior art in the domain of pilot training in that the present invention provides long-term effects on pilots which are data-driven. Brain performance is enhanced by learning/training rather by using external stimuli. It should be emphasized that any type of learning/training provides the effects, which last a prolonged period while the effects of the external stimuli are limited by a very short time. The feedback provided by the system of the preset invention relates to the current brain state of the tested trainee. From the abovementioned feedback, the trainee can learn how to control the level of activation of these specific brain regions with their own will. These changes are saved in the brain the same way as in any learning process.

The system of the present invention is configured for recording electroencephalographic signals relating to activation of particular brain regions related to movement such as the primary motor cortex. This is the reason for the use of high-quality EEG hardware—to ensure optimal data collected. By collecting this unique type of data from the user, analyzing it online and offline, we create a training environment that is highly personal which drives the user to increase their performance.

Generally, the system of the present invention is functioning as a Brain-Computer Interface (BCI) for pilot training and includes the following elements: (1) a Brain signal recording arrangement, (2) real-time signal analysis software, and (3) a user's front end (training environment in a cockpit representational form).

Reference is now made to FIG. 1 presenting a schematic diagram of system for testing and training a brain capability of planning and executing piloting activities and manoeuvres. Numeral 10 refers to an trainee to be tested. System 100 comprises memory unit 50 storing instructions for processing unit 40. The aforesaid processing unit is connected to electroencephalographic sensor arrangement 20 attachable to a head of said trainee such electroencephalographic signals generated on the head surface of trainee 10 are detectable. Trainee 10 is instructed to imagine executing a predetermined flight control action 15 in response to displaying a visual stimulus on display 30. Displaying the visual stimulus to trainee 10 is performed concurrently with measuring electroencephalographic signals on electroencephalographic sensor arrangement 20. According to the instructions stored in memory unit 50, processing unit calculates a concentration index, a motor control index and an alertness index (described in in detail below). Then a feedback message characterizing at least one said concentration, motor control, alertness and flight readiness are provided to trainee 10. The feedback in the form of a static avatar, a dynamic avatar, a text message, a sound pattern, a tactile pattern is in the scope of the present invention.

Referring to FIG. 2a, an exemplary electroencephalographic cap is made of an elastic synthetic fabric that comes in various sizes. The aforesaid cap holds the sensors exactly above the brain regions of interest. The standard EEG cap is known as the “10-20 system”. The “International 10-20 system” is a recognized method to describe the scalp electrodes' location. This standard testing system ensures a subject's study outcomes (clinical or research) could be compiled, reproduced, and effectively analyzed and compared using the scientific method. The system is based on the relationship between the location of an electrode and the underlying area of the brain, specifically the cerebral cortex.

Referring to FIG. 2b, in the electroencephalographic sensor arrangement, each EEG sensor is recording the electric fields that are underneath it. The neurons communicate with one another with changes in electric charges. It creates a difference in the electric field around them. That is the reason we can decode the brain's functions by analyzing these changes. The electroencephalographic sensor arrangement comprises sensors attachable at:
(1) frontal zone electrode locates on the midline of the frontal lobe
(2) parietal zone electrode locates on the midline of the parietal lobe;
(3-5) sensorimotor zone electrodes attached to the motor cortex.

In addition, ground and reference sensors are used to collect the signal.

The surface under the sensors is recommended to be cleaned prior to measurement and brought in electric contact the skin surface by means of a conductive gel in order to maximize signal-noise ratio, if necessary.

Referring to FIG. 2c, the sensors are connected to an amplifier that increases the electric signal's magnitude. The amplifier is attached to the cap using a cord. It transmits the amplified electroencephalographic signals to a computer, smartphone, or tablet connected by a USB connector using a wireless connection. Most of commercially available EEG signal amplifiers are usable in the present invention.

The EEG's data is streamed to the processing unit and structured with software according to a pre-set sensor montage. Referring to FIG. 2d, at the next step noise removal features are applied, such as removing the electric network's static electric disturbances (notch filter). The software allows the user to examine each sensor's connectivity level, which will enable them to add more gel if the impedance is too high or detect any sensor malfunctioning.

The EEG data is being streamed using LSL protocol (Lab Streaming Layer), picked up by the data-analyzing code.

Reference is now made to FIG. 3 presenting method 200 of for testing and training a brain capability of planning and executing flight control actions. Method 200 starts with providing system 100 (FIG. 1) for testing and training a brain capability of planning and executing piloting actions described above (step 210). After instructing said trainee to imagine executing the predetermined pilot action in response to displaying a visual stimulus (step 220), the aforesaid visual stimulus is displayed to the trainee to be tested concurrently with measuring electroencephalographic signals on the electroencephalographic sensor arrangement (step 230). The obtained electroencephalographic signals are processed and the concentration index, motor control index and alertness index are calculated (step 240). On the basis of the calculated indexes, a feedback message characterizing at least one of concentration, motor control, alertness and flight readiness (step 250). Steps 220-250 are recurred if needed.

EEG data is received over an LSL socket. The data analysis includes filtration. The EEG raw-data is analyzed in time windows with a shift (e.g., 500 or 1,000 samples with 50% shift) in each cycle. The goal of the analysis is to extract relevant brain function features that contribute to successful performance of motion control of cockpit and pilot controls.

The level of a person's concentration and activity level in the motor cortex can be deducted from fluctuations of the power of certain frequency bands. In the present system it is detected with at least five scalp electrodes.

Calculating of the “concentration” (or—brain engagement) index is performed according to the following algorithm:

Raw data is first filtered using an IIR filter, with half-power frequencies for a frequency range [alpha: 8 to 11 Hz; beta: 16-22 Hz; and theta 4-7 Hz] on the data from sensors attached to parietal zone (alpha) and frontal zone (beta and theta). We calculate power of each frequency band (alpha, beta, and theta) using a “bandpower” method based on the relevant filtered data. The concentration index for each cycle is the ratio of the powers of beta, theta and alpha.

Threshold—accuracy: At the beginning of each session, the system will determine a baseline that characterizes each trainee. The pilot trainee will sit still in front of an instructed flight activity or simulation for several minutes (2 minutes by default) to create an open-eyes baseline. The system collects the indices during the baseline collection and is used to set an trainee actualized threshold which would set the user's customized boundary. If exceeded, the system can determine that the user's concentration is high enough to give the trainee positive feedback.

According to an exemplary embodiment of the present invention, the aforesaid threshold can be set as a sum of values of lower bound and a compound of difficulty level and difference between upper and lower bounds

Upper bound is the average of the indices. Lower bound is the average minus two standard deviations of the index, and the Difficulty level is a value set by the user to adjust the challenge level to the user's abilities.

Training using aircraft controls feedback. During flight, the indices are continuously calculated and compared with the pilot trainee's threshold baseline through the backend module. Suppose the concentration index's current value exceeds the threshold set for it. In that case, the module will send the flight simulation a message) to grant positive user feedback. As an example, in a take off, landing or in flight procedure, the user will notice take off, landing or in flight procedure execution through shrinkage of a circle indicating the target, making the piloting and aircraft handling procedure more accurate. After several attempts during which the user exceeded the threshold in the current trial (for example, in a landing approach to a runway within a predetermined number of seconds), intermittent feedback is given (a satisfactory and safe approach resulting from appropriate throttle and flaps coordination).

The algorithm of calculation of the motor control index is the following:

First, raw data is filtered using an IIR filter, with half-power frequencies for a frequency range [Mu: 12 Hz to 15 Hz] for sensorimotor zone sensors, such as C3, Cz and C4 above the motor cortex. The bandpower function calculates the power of the Mu frequency band and the filtered data from these channels. The index from sensorimotor zone sensors, such as C3 and C4 is later used to evaluate current Mu desynchronization which is an action preceding movement.

Threshold—power exerted on the flying joystick manual instrument: all Mu power indices calculated during the acquisition of open-eyes baseline data from the sensorimotor zone, such as C3 and C4 electrodes are used to evaluate the trainee pattern of Mu rhythm of the specific user.

For example, the average Mu power of locations C3 and C4 (above the left motor cortex that controls the right-side limbs and vice versa) is used as the motor brain activity threshold.

Training on the basis of operation or procedure feedback: during the operation or procedure the current Mu power is continuously compared with the average value of the data collected in the open-eyes baseline. Specifically, the feedback is defined as positive if the sum of momentary Mu power and compound of difficulty lever and STD of MU at baseline is smaller than the mean MU at baseline.

“Mu power” represents the momentary Mu value; Difficulty level is a value set by the user to adjust the challenge level to the user's abilities; STD of Mu at baseline is the standard deviation of all Mu indices collected at open-eyes baseline. Suppose the current Mu power plus a portion of the standard deviation is lower than the average Mu at baseline. In that case, there is an activation of the motor cortex- and positive feedback is given.

In pilot training, the circular bar is gradually becoming full, and the color changes from red to green (via yellow and orange). The fill of the bar indicates to the user how to perform the neural action of activating these regions better. If the user succeeds in “charging” this bar during one trial, then the power exerted on the joystick or other manual instrument control would be strong enough to complete the predetermined flying action. Similar criteria are applicable to training and selection in all branches of commercial flying requiring quick and decisive accuracy Ability to mobilize physical vigor and mental alertness is also crucial in the training process for different types of aircraft, light aircraft, propeller, jet, cargo, airliners, gliders, and helicopters.

Only if the user could control both types of feedback (motor control and concentration) simultaneously to a sufficient degree-then the avatar (the pilot on the screen) will successfully complete the procedure.

The algorithm of calculation of the alertness (sleep) index is the following:

The power of the alpha band in the central parietal sensor, such as Pz, is collected at closed-eyes baseline (several minutes (2 minutes by default), immediately after open-eyes baseline collection) and compared to the alpha power open eyes baseline. Alpha power at this location is known as relating to the user's level of arousal; higher alpha power is usually associated with tiredness.

Threshold-Sleep detection: A moving average for both open-eyes and closed-eyes baseline data is calculated and compared. First, it is based on averaging of one time-window, then the interval increases until the moving average of closed eyes is at least higher in 2 standard deviations than the alpha values at open-eyes state. The moving average with the minimal time interval size that satisfies this condition is set as the threshold for “sleep detection”.

Feedback: During the surgical procedure, the current alpha power in the central parietal sensor or other appropriate location is continuously compared to the threshold. In the case of a landing the pilot makes an error such as an overshoot, the procedure on the screen is incomplete, an alert is given and a voice message may call out “Hey! Wake up, Danger!”.

Using data from early adopters in the field, advanced ML tools can be used, such as clustering algorithms, SVM classifiers, and Artificial Neural Networks to create a powerful pattern detection mechanism that would be highly user-specific, robust to EEG noise and would provide users with rich data-driven insights.

The final element of the system the present invention is the training environment for the user. The interface exposes the user to a real-time representation of his brain activity. It is essential in a neurofeedback learning process and is closing the loop that began in the brain data acquired using the EEG. We designed the products to allow pilots to train brain functions that are crucial to improve their performances. Thus far, we presented the underlying brain processes that our system tracks-motor imagery under high concentration levels. These brain processes are shared between different kinds of flying under different conditions in different aircraft. However, current research indicates that a feedback environment for this kind of neural training is much more efficient in a proper learning environment. Therefore, for each type of flying, unique virtual environments are be developed. The goal is to allow pilots to train in a familiar environment, which will enable them to transfer their learning efficiently into the ‘real-world’ of flying and controlling an aircraft.

The interface segment of the present invention is a computer-game environment designed to be highly contextual. For example, our first environment is an intestinal operation where a task might be to excise a growth in the intestine and suture the intestine securely, safely and effectively.

Reference is now made to FIGS. 4a to 4i presenting an exemplarily changing visual stimulus with improving the tested index. Specifically, the pilot is instructed to sit still, focus on the monitor, and imagine that he or she is executing a landing on the screen. For each attempt, the system gives the pilot a predetermined number of seconds. The neural activity in the case of real and imaginary flight activities is very similar. If our system detects strong activation on specific bands in specific brain areas related to this action, an indicator of power exerted upon the joystick or other manual flight control will increase (image below). This correlation between neural activation and a symbol presented on the screen allows the user to gain intuitive control of the activity of this neural network that is most important in controlling the operating arm and hand during the controlling of the joystick procedure.

With practice, the user learns how to control these brain areas, but he is also inducing changes in the way they are ‘wired’. The brain is an organ that changes continuously, neuron connectivity changes, networks of neurons are created or enhanced when constant activation takes place. These changes are the building blocks of learning, and they are the reason we get better with training and repetition.

As the player pilot imagines the action of taking off through, for example, a lively cross wind, the pilot receives continuous feedback on this activity. High activation is indicated by the fill and color of the circular bar (8 is still empty, and 0 is full power), the digit in the middle of the circle is the countdown, telling the user when the irreversible “flaps down” should begin. Only when the user can reach sufficient power—the avatar will have a chance to take off.

The task of activating these networks with repetitive training using our brain-trainer leads to similar actual, in-the-field activity, but does not suffer from many disadvantages of traditional practice.

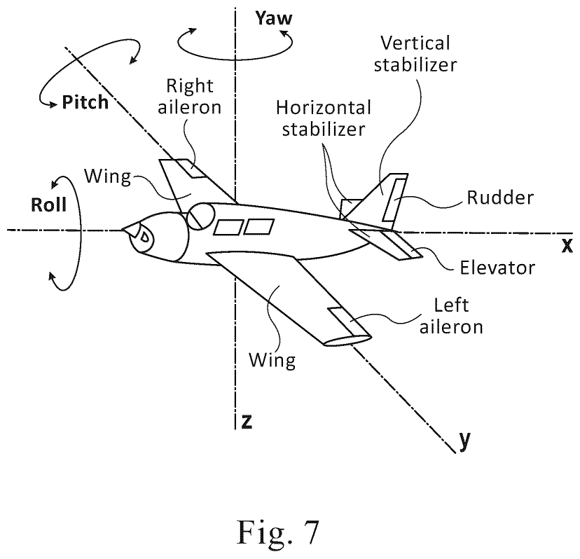

Reference is now made to FIG. 7 presenting another embodiment of the present invention, which could be maintaining a safe course in a turbulence event, or when an engine fails. The pilot can control in this game is how much power to distribute to the remaining engines and compensating wing lift. The accuracy is controlled by the level of engagement and focus of the player. When the system recognizes that the user is focused—a circle around the target will gradually lock on the target. Only when the white circle is locked on the target—will the remaining engine power arrive at the required level.

This way, the user gets ongoing feedback regarding two mental processes that he could not sense otherwise. The second form of feedback is referred to as ‘intermittent feedback’—the result of their efforts—if they managed to control both types of input to a sufficient level—the operative manual pressure on the surgical instrument that will be taken at the end of the predetermined time allocation will be successful, and the flight control mediated action would achieve the intended result. If only one condition is satisfied, then the pilot fails. If the pilot could not reach a sufficient concentration level during this trial then the flight control action would be inaccurately executed. If the pilot could not exert enough power by the motor imagery attempts—the flight would become unstable and failure may ensue.

With time and training, the vast majority of users learn how to modulate these neural processes by their own will, leading to important changes in neural networks involved in the pilot's performance.

If the pilot reaches sufficient power and accuracy—the avatar will complete the flight control procedure and the trainee will be alerted. Successfully completing a crucial flight operation is very rewarding for the pilot. In the brain, a general ‘rule’ is that neural activities that lead to reward will result in stronger connectivity and an increase in resources related to this activity. These changes will increase the pilot speed and accuracy in performing motor actions, providing subjective confidence and objective performance improvement.

At the end of the session, the pilots can observe their performance and track changes in their ability to concentrate or activate the brain's motor areas in the progression of the operation.

The interface will allow the pilot instructors to design the training session-how many repetitions, locations, complications, simulated emergencies are presented. Later the trainee pilot and the instructor can observe the performance of the pilot n achieved in the recent session, compare it to past sessions or other important analyses using a specially designed interface that is presented at the end of a session. It shows statistics based on the results of the training. It allows the pilots under training and the instructors to have the overall picture of the pilot's abilities and improvement using our trained brain-data.

Thus, the present invention provides the system and method for testing and training a brain capability of planning and executing motion activity. It should be emphasized that the disclosed invention is usable in any kind of human motion activity such as sport, surgery, aviation, post-traumatic and post-disease rehabilitation.

1. A system for testing and training a brain capability of a trainee for planning and executing motion activity; said system comprising:
a. an electroencephalographic sensor arrangement attachable to a head of said trainee;
b. a processor configured for receiving and analyzing electroencephalographic signals obtained from said trainee;
c. a memory storing instructions when executed by said processor for
i. instructing said trainee to imagine executing said motion action;
ii. measuring electroencephalographic signals on said electroencephalographic sensor arrangement;
iii. calculating at least one characteristic selected from the following:
1. a concentration index;
2. a motor control index;
3. an alertness index;
iv. providing said trainee with a feedback pattern based on at least one of the following indicators: an accuracy of the motion action based on said concentration index, a power of the motion action based on said motor control index, or motion readiness based on said alertness index;
v. iterating steps ii to iv; and
vi. generating a dynamic visual representation of the motion action being executed by an avatar in a virtual environment according to at least one of the power, accuracy, and motion readiness obtained during the iterations.

2. The system according to claim 1 comprising a display configured for providing a visual stimulus to said trainee, and said memory includes instructions for presenting a visual message and/or playing an audio message instructing the trainee to imagine executing said motion activity in response to displaying said visual stimulus.

3-4. (canceled)

5. The system according to claim 1, wherein said memory comprises an instruction of calculating said concentration index as a ratio of change of electroencephalographic signals at parietal-zone and frontal-zone electrodes at alpha, beta- and theta-frequencies obtained from said electroencephalographic signals at parietal-zone and frontal-zone electrodes measured at rest.

6. The system according to claim 1, wherein said memory comprises an instruction of calculating said motor control index as a ratio of change of electroencephalographic signals at sensorimotor zone electrodes, at Mu-frequency obtained from said trainee in response to a visual stimulus presented on a display below said electroencephalographic signals at sensorimotor zone electrodes measured at rest.

7. The system according to claim 1, wherein said memory comprises an instruction for calculating said alertness index as a ratio of change of electroencephalographic signals at parietal-zone electrode at alpha-frequency obtained from said trainee with open eyes over said electroencephalographic signals at parietal-zone with closed eyes.

8. The system according to claim 1, wherein said memory comprises an instruction of analyzing at least one of said concentration index, motor control index and alertness index of said trainee or a group of said trainees and presenting training progress data in a chronological manner.

9-11. (canceled)

12. The system according to claim 1, wherein said visual environment is selected from the group consisting of a soccer stadium, a baseball stadium, a basketball hall, a rugby stadium, an athletic stadium an operating theatre, dental operating room, in situ emergency environment or any surgical environment an aircraft cockpit, unmanned airborne vehicle control center, flight control center.

13. A method of testing and training a brain capability of a trainee to plan and execute motion activity; said method comprising steps of:
a. providing said system according to claim 1 for testing and training a brain capability of planning and executing motion activity;
b. instructing said trainee to imagine executing said motion action;
c. measuring electroencephalographic signals on said electroencephalographic sensor arrangement;
d. calculating said concentration index, motor control index and alertness index;
e. providing said trainee with a feedback pattern based on at least one of the following indicators: an accuracy of the motion action based on said concentration index, a power of the motion action based on said motor control index, and motion readiness based on said alertness index;
f. iterating steps f to h; and
g. generating a dynamic visual representation of the motion action being executed by an avatar in a virtual environment according to the at least one of the power, accuracy, and motion readiness obtained during the iterations.

14. The method according to claim 13, comprising a step of providing a display configured for providing a visual stimulus to said trainee, and generating an audio and/or visual message for instructing said trainee to imagine executing said motion activity in response to displaying said visual stimulus.

15-16. (canceled)

17. The method according to claim 13, wherein said step of calculating said concentration index comprises calculating a ratio of change of electroencephalographic signals at parietal-zone and frontal-zone electrodes at alpha-, beta- and theta-frequencies obtained from said trainee in response to a visual stimulus presented on a display over said alpha-, beta- and theta-frequencies obtained from said electroencephalographic signals at parietal-zone and frontal-zone electrodes measured at rest.

18. The method according to claim 13, wherein said step of calculating said motor control index comprises calculating a ratio of change of electroencephalographic signals at sensorimotor zone electrodes at Mu-frequency obtained from the trainee-in response to a visual stimulus presented on a display below said electroencephalographic signals at sensorimotor zone electrodes measured at rest.

19. The method according to claim 13, wherein said step of calculating said alertness index comprises calculating a ratio of excess of electroencephalographic signals at parietal-zone electrode at alpha-frequency obtained from said trainee with open eyes over said electroencephalographic signals at parietal-zone with closed eyes.

20. The method according to claim 13, comprising a step of analyzing at least one of said concentration index, motor control index and alertness index of said trainee or a group of said trainees and presenting training progress data in a chronological manner.

21-22. (canceled)

23. The method according to claim 13, wherein said feedback pattern relates to a visual environment selected from the group consisting of a sports motion action, a surgical action or a flight control action further wherein said motion action is an action directly concerned with executing a sports activity, a surgical manual or physical action, a flight control, joystick, rudder or other flight motion activity or any activity requiring physical motion of the limbs and eye coordination.

24. The method according to claim 13, wherein said visual environment is selected from the group consisting of a soccer stadium, a baseball stadium, a basketball hall, a rugby stadium, an athletic stadium an operating theatre, dental operating room, in situ emergency environment or any surgical environment, an aircraft cockpit, unmanned airborne vehicle control center, flight control center.

25. The method according to claim 13, comprising steps of calculating an integral index of sports, surgical or flight control readiness as a compound of at least two indexes selected from the group consisting of said concentration index, motor control index and alertness index and normalized by a sum thereof.

26. The system of claim 1, wherein said memory includes instructions for feeding said electroencephalographic signals into a machine learning model, and obtaining detected patterns robust to EEG noise from the machine learning model, wherein at least one of the concentration index, the motor control index, and the alertness index, is computed according to the detected patterns robust to EEG noise.

27. The system of claim 1, wherein said memory includes instructions for monitoring connectivity level of each sensor of the electroencephalographic sensor arrangement, and generating an indication for adding more gel.

28. The system of claim 1, wherein said memory includes instructions for:
computing a baseline threshold based on at least one of the concentration index, the motor control index, and the alertness index calculated during a baseline collection time interval;
wherein the feedback pattern is generated according to at least one of the concentration index, the motor control index, and the alertness index calculated during the iterations relative to the baseline threshold.

29. The system of claim 1, wherein said memory includes instructions for dynamically adapting the feedback pattern according to at least one of the calculated concentration index, the motor control index, and the alertness index, compared to a set difficulty level indicating a challenge level to the trainee's abilities.
